(19) United States
(12) Patent Application Publication
(10) Pub. No.: US 2014/0350327 A1
(43) Pub. Date: Nov. 27, 2014

(54) Device and method for interfering with sympathetic chain signaling for attenuating hot flashes, post-traumatic stress disorder, pain and dysautonomia

(71) Applicant: THE BOARD OF TRUSTEES OF THE LELAND STANFORD JUNIOR UNIVERSITY, PALO ALTO, CA (US)

(72) Inventors: Ada Shuk Yan Poon, Redwood City, CA (US); Vijaykumar Rajasekhar, Apple Valley, CA (US); Kathryn Hammond Rosenbluth, San Francisco, CA (US); Xiang Mackey Qian, Union City, CA (US); Scott Delp, Stanford, CA (US); John Paderi, San Francisco, CA (US); Tahel Altman, Ramat Hasharon (IL)

(73) Assignee: THE BOARD OF TRUSTEES OF THE LELAND STANFORD JUNIOR UNIVERSITY, PALO ALTO, CA (US)

(21) Appl. No.: 14/285,571

(22) Filed: May 22, 2014

Related U.S. Application Data
(60) Provisional application No. 61/826,394, filed on May 22, 2013.

(51) Int. Cl. A61N 1/36; A61M 5/172; A61N 2/00; A61F 7/12

(52) U.S. Cl. CPC A61N 1/36103; A61N 1/36139; A61F 7/12; A61M 5/1723; A61N 2/004
USPC 600/9; 607/117; 607/62; 607/59; 607/113; 607/72; 604/93.01; 604/65

(57) ABSTRACT

Provided herein are a device and method for attenuating posttraumatic stress syndrome, menopause symptoms, dysautonomia and pain by interfering with sympathetic chain signaling, particularly by blocking stellate ganglion conduction.

### 1. CROSS-REFERENCE TO RELATED APPLICATION

This application claims priority and other benefits from U.S. Provisional Patent Application Ser. No. 61/826,394 filed May 22, 2013, entitled “Device and method for interfering with the sympathetic chain for the treatment of hot flashes, PTSD, and pain syndromes”. Its entire content is specifically incorporated herein by reference.

### 2. TECHNICAL FIELD OF THE INVENTION

The present invention relates to a device and method for attenuating posttraumatic stress syndrome, menopause symptoms, pain and dysautonomia by interfering with sympathetic chain signaling, particularly by blocking stellate ganglion conduction.

### 3. BACKGROUND

Disturbances of the sympathetic nervous system, including menopausal symptoms, post-traumatic stress disorder and pain, can greatly affect the capacity of an individual to socialize, function and feel adequate in society.

The present invention addresses non-hormonal treatment options for such disturbances.

### 4. SUMMARY OF THE INVENTION

In one aspect of the present invention, a device is provided for attenuating symptoms in a subject suffering from a sympathetic nervous system disturbance by interfering with sympathetic chain signaling at cervical or thoracic levels. In various embodiments of the invention, the device comprises an interface for interfering with sympathetic chain signaling at cervical or thoracic levels, an actuator for modulating sympathetic chain signaling, a power source for providing power to said actuator, optionally one or more sensors for detecting changes, optionally controls for capturing information from said one or more sensors and for integrating said information into parameters and optionally a medium for storing said parameters in digital format. In the various embodiments, the interfering can be of electric, thermal or magnetic nature.

In another aspect of the invention, a method is provided for attenuating symptoms in a subject suffering from a sympathetic nervous system disturbance by interfering with sympathetic chain signaling at cervical or thoracic levels. Such disturbances encompass menopausal symptom including hot flashes, posttraumatic stress disorder, pain and dysautonomia.

The above summary is not intended to include all features and aspects of the present invention nor does it imply that the invention must include all features and aspects discussed in this summary.

### 5. BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 shows a device with a surgically implanted interface (100) in immediate proximity of the stellate ganglion.

FIG. 2 shows a device with an external interface (110) that interferes transcutaneously with the stellate ganglion and sympathetic chain.

FIG. 3 shows a device with an external interface that may be shaped (120) for positioning close to the stellate ganglion, T1, T2 or lower (130) at T3, T4.

FIG. 4 shows various embodiments of the method to attenuate disturbances in the sympathetic nervous system. (A) shows the implanted device (110) in close proximity to the stellate ganglion; here the implant is powered by a battery (140) which is implanted as well. (B) shows the implanted device (110) in close proximity to the stellate ganglion; here the implant is powered by a wireless external device (150).

### 6. DETAILED DESCRIPTION

Provided herein are systems and methods for attenuating disturbances in the sympathetic nervous system. Before describing detailed embodiments of the invention, it will be useful to set forth definitions that are utilized in describing the present invention.

### 6.1. Definitions

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The term “attenuate”, “attenuation”, as used herein, means to reduce or to lessen in intensity and/or frequency.

The term “subject”, as used herein, refers to a mammal, preferably a human.

The term “block” or “blocking”, as used herein, refers to disrupting or inhibiting the transmission of neuronal, nerve impulses within the sympathetic nervous system.

### 6.2. The Sympathetic Chain

The sympathetic chain is composed of inferior, middle and superior cervical nerve clusters known as ganglia extending alongside the spinal column forming the sympathetic nervous system.

The stellate ganglion, which is also known as the cervicothoracic ganglion, is formed through a fusion of the inferior cervical ganglion with the first thoracic ganglion and is located in front of the neck of the first rib. The stellate ganglion, as part of the sympathetic nervous system, innervates the face, neck, arm and upper chest. The stellate ganglion extends into the space between the first thoracic (T) vertebral bodies and the C7 vertebrae. Nerve fibers that supply the head and the neck originate from the 1st and 2nd thoracic spinal segments, whereas nerve fibers supplying the upper extremities originate from the 2nd through the 9th thoracic segments.

The sympathetic nervous system is composed of central and peripheral components and utilizes an array of ascending and descending communication channels which make it possible to exert far-reaching effects.

Sometimes, when sympathetic nerve fibers are upregulated or sensitized by trauma or other stimuli, the overactive sympathetic activity can cause various conditions including hot flashes, chronic, post-traumatic stress disorder, hyperhidrosis, Raynaud's phenomenon.

Nerve fibers associated in the sympathetic output of hot flashes in the head, trunk, and arms pass through the stellate ganglion. Furthermore, thoracic vertebrae T2-T4 of the sympathetic chain are also involved in the sympathetic outflow of hot flashes to the upper body. T2 sympathectomy reduces craniofacial hyperhidrosis and can reduce flushing and sweating in patients with Harlequin syndrome, a rare disease with unilateral flushing and sweating in the face, upper body and arms.

Stellate ganglion block and sympathetic chain block. In general, the stellate ganglion and sympathetic chain can be blocked by an injection of a local anesthetic to the neck where the stellate ganglion and sympathetic chain are located. However, blocking the stellate ganglion and sympathetic chain with local anesthetics has its limits. The local anesthetic is short lasting and does not provide long term benefits; the block itself can be complicated by severe adverse events including hematoma, total spinal shock and infection.

### 6.3. Disturbances Of The Sympathetic Nervous System

Hot flashes. Menopause symptoms hallmarked by recurrent hot flashes occur in up to 75% of women undergoing natural menopause and can persist for years. The symptoms of hot flashes include sudden sensations of intense heat with sweating, flashing, and peripheral vasodilatation. Severe hot flashes, which can cause tachycardia, diaphoresis, nausea, dizziness, anxiety, headache, and weakness, also substantially increase the risk of sleep deprivation, depression, sexual dysfunction, and other serious medical conditions. Hot flashes are especially problematic in women who have survived breast or ovarian cancer because chemotherapy or surgical removal of reproductive organs can lead to the premature occurrence of menopause. The occurrence of hot flashes in breast cancer survivors is often significantly more frequent, severe, distressing, and of greater duration than in women who did not require cancer treatment.

Current treatment options for hot flashes. Current treatment options for hot flashes have varying degrees of effectiveness. Available options include: hormone replacement therapy (HRT), herbal remedies, and non-hormonal pharmaceuticals including anti-depressants. HRT is by far the most commonly used treatment for hot flashes, reducing frequency of hot flashes by 60% to 85%. However, studies from the National Institutes of Health and the Women's Health Initiave brought to light that prolonged hormone therapy posed more health risks than benefits, especially for women who have a genetic disposition to develop female hormone-dependent cancers such as breast and ovarian cancer. Because of the undesired side effects including heart attack, blood clot, and increased incidence of breast cancer recurrence associated with hormone therapy, today only about 1 in 5 post-menopausal American women resort to HRT. Non-hormonal alternatives and safer treatments for hot flashes are thus an extremely active area of research and therapy development.

Pain. The sensation of pain functions as a natural warning sign that an injury has occurred and is meant to trigger a protective response. In many cases, however, the sensation of pain remains as persistent chronic inflammatory or cancer pain and becomes debilitating both physically and psychologically. Almost one in five adult Americans experiences chronic and persistent pain and seek treatment for chronic pain each year. Complex regional pain syndrome (CRPS), formerly reflex sympathetic dystrophy (RSD), “causalgia”, or reflex neurovascular dystrophy (RND) are examples of chronic pain. Besides severe pain, they are characterized by swelling and changes in the skin, often time affecting an arm or a leg and often spread throughout the body. Treatment is complicated, involving medications, physical therapy, psychologic treatments, and spinal cord stimulator (SCS), if all other therapy fails.

Approved by the FDA in 1989, SCS has become a standard treatment for patients with refractory chronic pain in their back and or extremities who have failed other treatments. SCSs are implanted by either neurosurgeons or pain physicians, in which wires with electrical leads on their tips are implanted through an epidural needle in the back near to the spinal cord dorsal column. The leads are then connected via a tunneled extension cable to a programmable pulse generator that is implanted in the upper buttock or abdomen (under the skin) or chest and emits stimulation electrical currents to the spinal cord dorsal column. In spite of its popularity, current studies show that only about 50-60% of patients who try SCS find meaningful pain relief (>50%).

In addition, SCS does not eliminate the pain; rather, it replaces the intense pain with the more tolerable paresthesia feelings. In addition, SCS implantation is an invasive procedure. It is associated with many complications and can even lead to devastating paralysis, nerve injury, and death. The most common complications include lead migration and hardware failure, followed by pain at the implantation site, and clinical infection. SCS is also expensive, costing on average from $20K to $60K depending on complications and perioperative visits.

Post-Traumatic Stress Disorder (PTSD). PTSD is an anxiety disorder caused by psychological trauma. This trauma may include rape, combat, neglect, physical abuse or other traumatic experiences. PTSD is differentiated from an acute stress response in that it endures longer than 30 days. Individuals suffering from PTSD periodically re-experience the traumatic event, engage in persistent avoidance behaviors, and are clinically distressed in daily activities such as social relations or occupational activities. Treatments for PTSD are lacking and generally include behavioral therapy and pharmaceutical treatment. Approximately 8% of Americans will suffer PTSD at some time in their life. Women are approximately twice as likely as men to suffer PTSD.

Dysautonomia is a general term used to describe various conditions that result from a malfunctioning of the Autonomic Nervous System (ANS), which is essential for maintaining homeostasis of human body, including heart rate, blood pressure, digestion, dilation, and temperature control.

Notable examples of dysautonomia include hyperhidrosis, in which dysautonomia results in inappropriately heavy perspiration, and Raynaud's phenomenon, in which blood flow to fingers decreases significantly in the cold and under emotional stress, leading to color changes, pain, and tissue hypoxia.

Approximately 2-3% of Americans suffer from hyperhidrosis. Hyperhidrosis is not life threatening but can profoundly impair social interactions and cause anxiety. The stellate ganglion and the No. 1-4 thoracic ganglia of the sympathetic chain are believed to play an essential role in the abnormal signal generation to sweat glands of the upper limb. Prevalence studies suggest that 3-12.5% of men and 6-20% of women report symptoms of Raynaud's phenomenon, with higher prevalence in colder climates. Typical symptoms of Raynaud's phenomenon are pain within the affected extremities, along with discoloration and paresthesia/numbness. Both hyperhidrosis and Raynaud's phenomenon are refractory to conventional treatments including Botox injection and vasodilators, and may require a sympathectomy in which the sympathetic nerves that innerve the blood vessels of the limb are surgically cut.

### 6.4. Device For Attenuating Symptoms In A Subject Suffering From A Sympathetic Nervous System Disturbance

Herein described is a device that provides novel ways of interfering with, i.e. manipulating, areas of the stellate ganglion and sympathetic chain to block nerve conduction, thus providing symptom relief in patients with hot flashes, minimizing pain associated with chronic pain syndrome, reducing the occurrence of episodes of PTSD, and alleviating severity of dysautonomia.

The mechanism of interference may be of thermal, magnetic or electric nature.

The device may be an implant or may be a non-invasive body surface device.

The device can be used for one-time applications or repeated applications, as is best suited to achieve a therapeutic effect, i.e. a lessening in the intensity and/or frequency of the targeted disturbance.

The device may be used in any combination of interface, actuators, power sources, delivery system or in the absence of any of these, as would be apparent to one of ordinary skill in the art in view of the teachings herein. An embodiment of the device is described in Example 7.2.

### 6.5. Method For Attenuating Symptoms In A Subject Suffering From A Sympathetic Nervous System Disturbance Using Above Described Device

In the broadest sense, a device and method are provided for attenuating symptoms, such as hot flashes, in a subject suffering from a sympathetic nervous system disturbance, by interfering with the sympathetic chain signaling, for example at the level of the stellate ganglion. The subject is generally a mammal and preferably a human.

Schedule of administration. The interference with sympathetic chain signaling, for example, at the level of the stellate ganglion, is maintained for a period of time sufficient to effect a therapeutically desired change, as evidenced by an attenuation, i.e. amelioration, of symptoms of hot flashes, pain or post-traumatic stress disorder. Such treatment may involve administering the interference at least once, or for about a few days or about a week; at least about two weeks; at least about 3 weeks; at least about one month; at least about two months; at least about four to six months; or longer, for example at least about one or more years. For extended treatment; e.g. treatment of one or more years, a schedule may involve intermittent periods, such as one week on and one week off; two weeks on and two weeks off; one week in a month, etc.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### 6.6. Assessing Improvement

An improvement of symptoms of a disturbance such as hot flashes can be assessed using thermal sensors within the described device and evaluating an improvement following treatment, i.e. attenuation, as described herein. By improvement is meant at least an amelioration of the symptoms, where amelioration is used to refer to at least a reduction in the magnitude and/or frequency of hot flashes.

### 7. EXAMPLES

The following examples are put forth to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### 7.1 General Design of a Device And Method For Interfering With The Sympathetic Chain

Here, a non-pharmacological, on demand, reversible method is described to interfere with the sympathetic chain via a wirelessly powered, electro-stimulation device implanted nearby the Stellate Ganglion. The stimulation electrodes deliver electrical stimulation to block the Stellate Ganglion from conducting and maintaining sympathetic tone. An external device able to generate electromagnetic fields for transfer energy is used to power up the stimulation electrodes in the implanted device wirelessly via the midfield energy transfer method.

In one embodiment, the stellate ganglion is electrically stimulated by low or high frequency biphasic pulses, which interferences nerve conduction and blocks its sympathetic function.

An exemplary process involves the following steps:

Step 1. Identify the Stellate Ganglion in the neck. This step is facilitated by a conventional method such as ultrasound, endoscopy, fluoroscopy to guide precise placement of the stimulation device. Step 2. An implantable device of miniature size with stimulation electrode on the device-housing surface is placed in immediate proximity to the stellate ganglion. An introducer needle of appropriate size is placed subcutaneously to the fascia right above the stellate ganglion. A small incision to the skin might be required to assist the placement. An accurate placement can be facilitated utilizing ultrasound, endoscopy assistance, or fluoroscopy. The stimulation device is then threaded through the introducer needle until it reaches the position just above or near the target. The introducer needle is then removed, leaving the stimulation device with the stimulation electrode in place above the Stellate Ganglion. In order to minimize the size, the implant device will be powered directly by energy transferred from an external device. Step 3. The circuit in the implanted stimulation device delivers bi-phasic stimulation pulse with 30-50 Hz up to 30-40 kHz directly to the stellate ganglion to block sympathetic signal conduction. An external device transfers energy to power up the implanted device with radiofrequency transmission frequency ranging from 1 MHz to 100 GHz. Step 4. A circuit is integrated in the implanted device to measure impedance between electrode and nerve/tissue, which is used to give feedback to a controller system that can subsequently adjust stimulation voltage and current when impedance varies due to time change and/or individual difference. Step 5. Patient is able to control and select various electrode stimulation combinations, such that an ideal control can be achieved.

### 7.2 Device And Method For Interfering With The Sympathetic Chain For Attenuating Hot Flashes

In embodiments of attenuating hot flashes using the device and method of the present invention, a device interferes with the sympathetic chain or branches off the sympathetic chain from the superior cervical ganglion down to T4 of the thoracic sympathetic chain or lower, i.e. to at least T5-T7. The device can comprise any of an interface, an actuator, a power source, one or more sensors, controls for capturing information, a medium for storing parameters.

Interface. An interface is the connection of the device to or near the stellate ganglion in the sympathetic chain. The function of the Interface is to provide physical, electrical and/or chemical interference with the cervical or thoracic sympathetic chain or branches off the cervical or thoracic vertebrae.

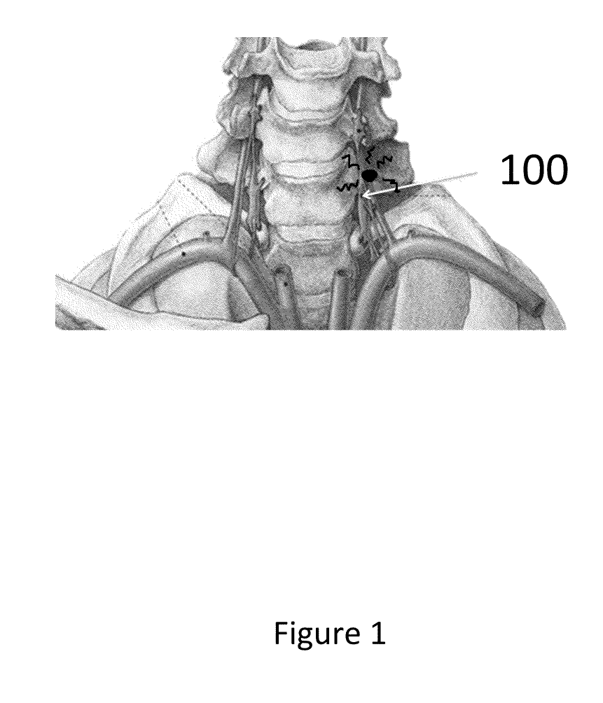

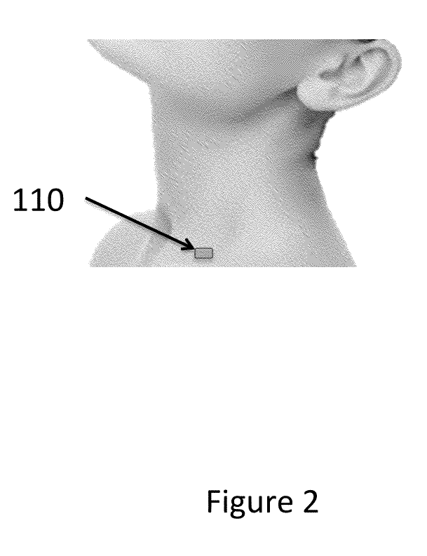

The Interface can be physically implanted at the stellate ganglion or in its immediate proximity, as shown in FIG. 1, or below the stellate ganglion, at the level or T1, T2, T3 or T4, or elsewhere along the sympathetic chain communicating with the ganglion. As shown in FIG. 2, the interface can be external to the body and interacting transcutaneously with the stellate ganglion or sympathetic chain.

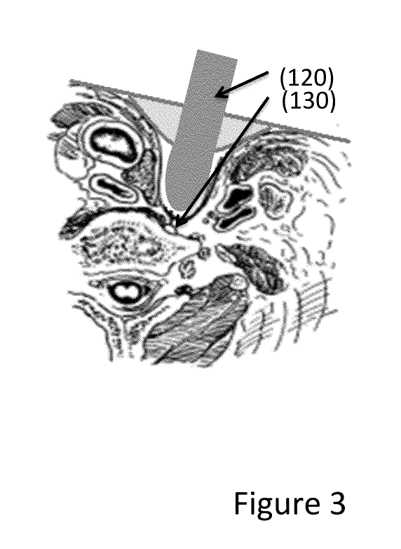

The Interface can be physically implanted at the stellate ganglion or in its immediate proximity, as shown in FIG. 1, or below the stellate ganglion, at the level or T1, T2, T3 or T4, or elsewhere along the sympathetic chain communicating with the ganglion (see FIG. 3). As shown in FIG. 2, the interface can be external to the body and interacting transcutaneously with the stellate ganglion or sympathetic chain.

In one embodiment, the Interface is an implantable chip, containing a set of electronic circuits, made from a biocompatible, rigid polymeric material or flexible material such as silicone. The chip may be expandable or inflatable such that it may be inserted in a collapsed configuration, such as through a needle, and expanded or inflated in position.

In another embodiment, the implant may include a nerve cuff to wrap around and maintain contact with a ganglion or branch (ramus) off the sympathetic chain. In another embodiment, the implant may be placed adjacent to a sympathetic ganglion, for example the Stellate, T1, T2, T3 or other cervical or thoracic sympathetic ganglions by means of anchoring to adjacent bones (such as the ribs), muscles (such as longus colli), nerves, vessels (such as the subclavian or vertebral artery or vein) or connective tissue. The bone may be, but is not limited to, a cervical vertebrate body. The muscle attachments may include, but are not limited to, the longus colli muscle surrounding the stellate ganglion. The attachment may include an adhesive material, a bone screw, anchoring barbs or other means of attaching implants.

In a further embodiment, the interface is an implant that is positioned by collapsing a lung to reach the sympathetic chain and the implant is placed in position on or in close proximity to the rami or ganglion of the thoracic or cervical sympathetic chain.

In another embodiment, the Interface is an external device that interferes transcutaneously with the sympathetic chain nerves or an implant. This interference may include electrical or thermal energy transfer. The external device may be shaped for positioning close to the stellate ganglion, T1, or T2, or T3, or T4 of the sympathetic chain by compressing and displacing adjacent anatomy as it is pushed in the direction of the sympathetic chain. This advancement may be guided by ultrasound.

In another embodiment, the interface is an intravascular device positioned in the blood vessels adjacent to the stellate ganglion or other regions of the sympathetic chain, including but not limited to the carotid, vertebral, and subclavian blood vessels. Intravascular embodiments of the interface include, but are not limited to intravascular stents or intravascular catheters.

Actuator. The actuator is the mechanism by which the device influences the sympathetic chain. It functions to modify sympathetic chain signaling, i.e. the propulsion of action potentials in the nerve fibers. This influence may be excitatory or inhibitory. Also, this influence may be acute and, thus, only temporarily block or reduce the flow or content of sympathetic chain information, for the influence can be longer term.

The actuator may act in a number of ways, including but not limited to activating, deactivating, or interfering with nerves that delivers sympathetic signals, some passing through the stellate ganglion.

In one embodiment, the actuator generates pulsed radiofrequency (RF) energy. The pulsed RF may be transcutaneous from the surface outside the human body or from an implant inside the human body. In just one example, PRF uses radiofrequency current in short (20 ms), high-voltage bursts; the “silent” phase (480 ms) of PRF allows time for heat elimination, generally keeping the target tissue below 42° C.

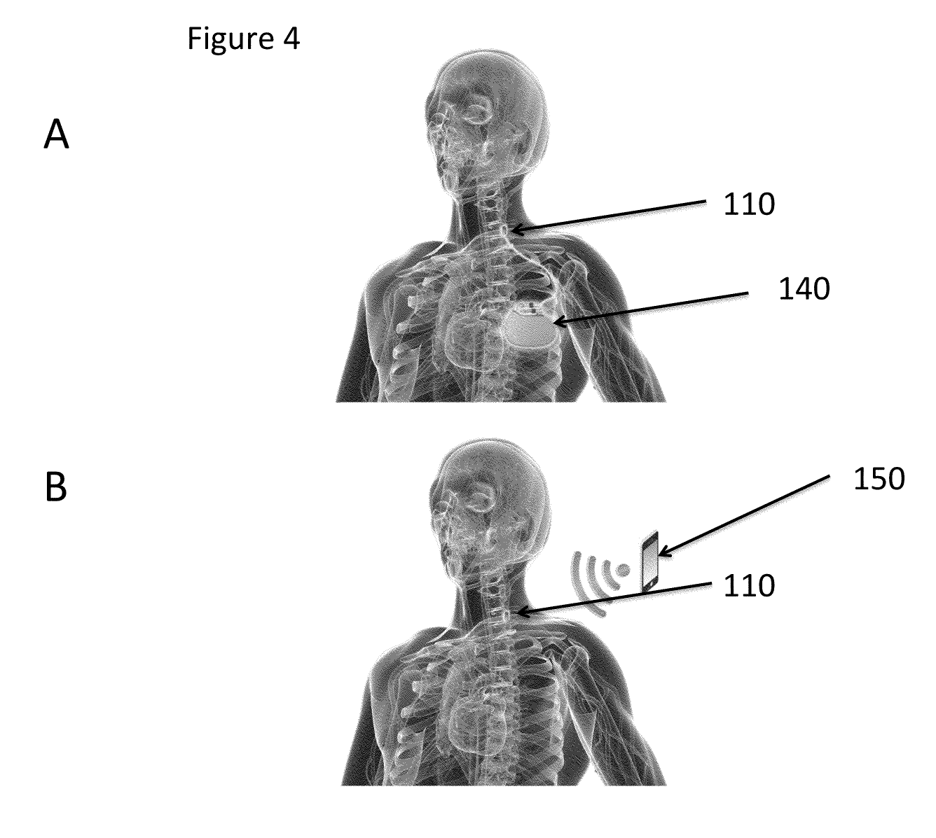

In another embodiment, the actuator includes two or more stimulation electrodes. These electrodes will be placed close to stellate ganglion and sympathetic chain, and will deliver electrical pulses to block the stellate ganglion and sympathetic chain from conducting and maintaining sympathetic tone. An external device that generates electromagnetic fields transfers energy to power up the implanted device wirelessly. The stimulator is a smart device that can be designed to include on-board processors, sensors, and wireless transceivers. It can be programmed wirelessly to change the frequency, duration, intensity, and shape of the stimulation pulse waveforms. The stimulation pulse waveforms can be programmed to be mono-phasic or bi-phasic. In some variations, the pulse frequency ranges from 10 Hz to 10 kHz. This invention involves the placement of the stimulation device near the stellate ganglion/sympathetic chain and subsequent wirelessly delivered electrical stimulation of stellate ganglion/sympathetic chain to directly block sympathetically mediated functions (see FIG. 4B).

In another embodiment, the Actuator delivers thermal energy. The thermal energy could have a non-ablative effect cooling the tissue down to 4 degree Celsius. It could be generated from the implant located close to the stellate ganglion,or T2 of the sympathetic chain, or other ganglion of the cervical or thoracic ganglion, or can be delivered externally from a source such as, but not limited to, a focused ultrasound generator. The thermal effect can be continuous or periodic.

In another embodiment, the Actuator both cools and stimulates the area of the sympathetic chain (anywhere from the superior cervical ganglion down to T4) until the target area of the sympathetic ganglion is blocked. The cooling may occur before, during, or after the electrical stimulation.

In another embodiment, the Actuator interferes with the nerve by exposing the nerve to a rapidly oscillating magnetic field. In another embodiment, the actuator interferes with nerves by electrical stimulation. Electrical or magnetic interference may excite the nerves or block them by depleting the chemical stores required for the neuron to fire.

In another embodiment, the Actuator interferes with the nerves by chemical excitation/block from an implanted or transcutaneous drug delivery port, pump, or other sustained release drug delivery mechanisms. As an example, some drugs that could be used to perform the chemical excitation/ block include, but are not limited to, local anesthetics such as lidocaine, ropivicaine, and other drugs known to interfere with sodium channels or calcium channels in nerves. Another example of chemical excitation/block is from alcohol-based chemicals that interfere with nerves.

Power source. The power source provides power for the actuator. The actuator can be powered wirelessly from an external power source, or can be powered internally from an implant, or draw energy from within the human body (see FIG. 4A). The power source can be a skin patch or a remote device powered by the user. The power may be from an electrical outlet or a battery source. Power can be transferred by radiofrequency coupling between the Power Source and Receiver in the implant. This power transfer could be in the radio spectrum, microwave spectrum or other spectrums.

The implanted device can include smart electronics to regulate the voltage received through the radiofrequency coupling. It may include a battery or capacitors to store energy on the device. It can include an electrically resistive component to heat adjacent tissue during excitation. It can include a magnetically susceptible material to enhance the local magnetic fields for electromagnetic excitation of the nerves.

Sensors. In another embodiment, the implant contains sensors. These sensors may include (i) thermal sensors for detecting a hot flash or for monitoring a successful attenuation of a hot flash, (ii) motion sensors for detecting sleep, (iii) electric listening sensors for detecting activity in the stellate ganglion or sympathetic chain, or (iv) flow sensors for detecting the effect of treatment, i.e. attenuation of a hot flash.

Controls/capture of sensed information. In another embodiment, the implant contains controls to integrate the information from the sensors into parameters for nerve excitation or inhibition.

Memory. In another embodiment, the implant contains a medium for storing information about the usage history, usage parameters and other information pertinent to the use of the device.

What is claimed is:

1. A device for attenuating symptoms in a subject suffering from a sympathetic nervous system disturbance by interfering with sympathetic chain signaling at cervical or thoracic levels, the device comprising
(a) an interface for interfering with sympathetic chain signaling at cervical or thoracic levels,
(b) an actuator for modulating sympathetic chain signaling,
(c) a power source for providing power to said actuator.

2. The device according to claim 1, furthermore comprising one or more sensors for detecting changes such as changes in temperature, movement, sympathetic neural activity or flow.

3. The device according to claim 2, furthermore comprising controls for capturing information from said one or more sensors and for integrating said information into parameters.

4. The device according to claim 3, furthermore comprising a medium for storing said parameters in digital format.

5. The device according to claim 1, wherein said interfering is of electric, thermal or magnetic nature.

6. The device according to claim 1, wherein said actuator delivers electrical pulses.

7. The device according to claim 1, wherein said actuator delivers thermal energy.

8. The device according to claim 1, wherein said actuator delivers a chemical effect of inhibitory or excitatory nature.

9. A device for attenuating symptoms in a subject suffering from a sympathetic nervous system disturbance by interfering with sympathetic chain signaling at cervical or thoracic levels, the device comprising
(a) an interface for interfering with sympathetic chain signaling at cervical or thoracic levels,
(b) an actuator for modulating sympathetic chain signaling,
(c) a power source for providing power to said actuator,
(d) one or more sensors for detecting changes,
(e) controls for capturing information from said one or more sensors and for integrating said information into parameters,
(f) a medium for storing said parameters in digital format.

10. The device according to claim 9, wherein said interfering is of electric, thermal or magnetic nature.

11. The device according to claim 9, wherein said actuator delivers electrical pulses.

12. The device according to claim 9, wherein said actuator delivers thermal energy.

13. The device according to claim 9, wherein said actuator delivers a chemical effect of inhibitory or excitatory nature.

14. The device according to claim 9, wherein said changes are changes in temperature, movement, sympathetic neural activity or flow.

15. A method for attenuating symptoms in a subject suffering from a sympathetic nervous system disturbance, comprising interfering with sympathetic chain signaling at cervical or thoracic levels using the device according to claim 1,
wherein said interfering is effective to achieve attenuation of said disturbance.

16. The method according to claim 15, wherein said disturbance is a menopausal symptom including hot flashes.

17. The method according to claim 15, wherein said disturbance is posttraumatic stress disorder.

18. The method according to claim 15, wherein said disturbance is pain.

19. The method according to claim 15, wherein said disturbance is dysautonomia.
